(19) United States
(12) Patent Application Publication
(10) Pub. No.: US 2022/0222853 A1
(43) Pub. Date: Jul. 14, 2022

(54) METHOD AND DEVICE FOR CALCULATING THE DISTANCE BETWEEN EYES AND THE EYES? TARGET

(71) Applicant: Industrial Technology Research Institute, Hsinchu (TW)

(72) Inventors: TSAI-YA LAI, New Taipei City (TW); SHIH-MING CHANG, Hsinchu City (TW); TING-HUI CHIANG, Taichung City (TW)

(21) Appl. No.: 17/557,717

(22) Filed: Dec. 21, 2021

Related U.S. Application Data
(60) Provisional application No. 63/128,877, filed on Dec. 22, 2020.

(30) Foreign Application Priority Data
Oct. 13, 2021 (TW) 110137874

(51) Int. Cl. G06T 7/77; G06V 10/774; A61B 3/14

(52) U.S. Cl. CPC G06T 7/77; G06V 10/774; G06T 2207/10028; G06T 2207/20084; A61B 3/14

(57) ABSTRACT

A method for calculating a distance between eyes and the eyes' target includes the steps of: inputting at least one image data of eyes and the eyes' target corresponding to at least one train object into a calculation module for establishing training data of an eye-distance measurement unit; utilizing a movable image capture module to obtain a set of image-capturing data of a test subject and the eyes' target; inputting the set of image-capturing data to the eye-distance measurement unit for analysis; based on the training data, utilizing the eye-distance measurement unit to mark out a set of three-dimensional coordinate values of the eyes and the eyes' target corresponding to the test subject; and, based on the set of three-dimensional coordinate values, utilizing the eye-distance measurement unit to calculate the distance between the eyes of the test subject and the eyes' target.

### CROSS REFERENCE TO RELATED APPLICATION

This application claims the benefits of U.S. provisional application Ser. No. 63/128,877, filed Dec. 22, 2020, and Taiwan application Serial No. 110137874, filed Oct. 13, 2021, the disclosures of which are incorporated by references herein in its entirety.

### TECHNICAL FIELD

The present disclosure relates in general to a method and device for calculating a distance, and more particularly to a method and device for calculating a distance between eyes and a target of the eyes.

### BACKGROUND

Myopia is one of common eye diseases. According to a report of the World Health Organization, people with myopia in the world is estimated to exceed 1.4 billion, and with visual impairment is up to 253 million. It is estimated that the number of visual impairment in the future will increase by three times. By 2050, there will be nearly 5 billion people with myopia in the world, of which 1 billion are at risk of blindness due to myopia.

Improper distancing to use eyes is one of key factors to the occurrence and deterioration of myopia. However, currently, there is no recognized record or reminder for such a problem. In the process of using eyes, including reading, writing, using 3C products, etc., individuals usually cannot detect the distance by their naked eyes, and so maintaining a proper eye-to-object distance effectively is actually hard to achieve.

In addition, while in facing a vision deterioration, the ophthalmologist is usually hard to provide any specific method or auxiliary device clinically other than an oral health promotion education. Namely, in the art, no specific or convincing advise in maintaining good visual distances according to reliable or practical data can be provided to the patient in urgent need or as a reference for adjusting treatment strategy.

Particularly, in a conventional automatic driving system, technical means of using image data for spatial identification or distance measurement are utilized, but empirically the relative error of the system would be between 15% and 149%. Definitely, such an error scale is too large to be suitable for calculating the distance between eyes and a target of the eyes.

Accordingly, how to provide a method and device for calculating a distance between eyes and the eyes' target has become a problem urgent to be solved in the art.

### SUMMARY

In one embodiment of this disclosure, a method for calculating a distance between eyes and the eyes' target includes the steps of: inputting at least one image data of eyes and the eyes' target corresponding to at least one train object into a calculation module for establishing training data of an eye-distance measurement unit; utilizing a movable image capture module to obtain a set of image-capturing data of a test subject and the eyes' target; inputting the set of image-capturing data to the eye-distance measurement unit for analysis; based on the training data, utilizing the eye-distance measurement unit to mark out a set of three-dimensional coordinate values of the eyes and the eyes' target corresponding to the test subject; and, based on the set of three-dimensional coordinate values, utilizing the eye-distance measurement unit to calculate the distance between the eyes of the test subject and the eyes' target.

In another embodiment of this disclosure, a method for calculating a distance between eyes and the eyes' target includes the steps of: inputting at least one image data of eyes and the eyes' target corresponding to at least one train object into a calculation module for establishing training data of an eye-distance measurement unit; utilizing a movable image capture module to obtain a first-capturing image data of a test subject and the eyes' target; varying an image angle or distance of the movable image capture module, and utilizing the movable image capture module again to obtain a second-capturing image data of the test subject and the eyes' target; inputting the first-capturing image data and the second-capturing image data to the eye-distance measurement unit for analysis; based on the training data, utilizing the eye-distance measurement unit to mark out a first set of three-dimensional coordinate values and a second set of three-dimensional coordinate values corresponding to eyes of the test subject and the eyes' target, respectively; and, based on the first set of three-dimensional coordinate values and the second set of three-dimensional coordinate values, utilizing the eye-distance measurement unit to calculate the distance between the eyes of the test subject and the eyes' target.

In a further embodiment of this disclosure, a device for calculating a distance between eyes and the eyes' target includes a movable image capture module and a calculation module. The movable image capture module is configured for imaging a test subject and the eyes' target to obtain a set of image-capturing data. The calculation module, having an eye-distance measurement unit, is configured for utilizing the eye-distance measurement unit to analyze the set of image-capturing data to further mark out a set of three-dimensional coordinate values corresponding to an eye of the test subject and the eyes' target. The eye-distance measurement unit evaluates the set of three-dimensional coordinate values to calculate the distance between the eye of the test subject and the eyes' target. In addition, at least one image data of the eyes and the eyes' target corresponding to at least one train object is inputted into the calculation module for establishing training data of the eye-distance measurement unit.

In one more embodiment of this disclosure, a device for calculating a distance between eyes and the eyes' target includes a movable image capture module and a calculation module. The movable image capture module is configured for imaging a test subject and the eyes' target to obtain a first-capturing image data, and further to obtain a second-capturing image data of the test subject and the eyes' target after varying an image angle or distance of the movable image capture module. The calculation module, having a eye-distance measurement unit, is configured for utilizing the eye-distance measurement unit to analyze the first-capturing image data and the second-capturing image data to further mark out a first set of three-dimensional coordinate values and a second set of three-dimensional coordinate values corresponding to eyes of the test subject and the eyes' target. The eye-distance measurement unit evaluates the first set of three-dimensional coordinate values and the second set of three-dimensional coordinate values to calculate the distance between the eyes of the test subject and the eyes' target. In addition, at least one image data of the eyes and the eyes' target corresponding to at least one train object is inputted into the calculation module for establishing training data of the eye-distance measurement unit.

Further scope of applicability of the present application will become more apparent from the detailed description given hereinafter. However, it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The present disclosure will become more fully understood from the detailed description given herein below and the accompanying drawings which are given by way of illustration only, and thus are not limitative of the present disclosure and wherein:

FIG. 1A is a schematic view of a first embodiment of the device for calculating a distance between eyes and the eyes' target in accordance with this disclosure;

FIG. 1B is a schematic view of a second embodiment of the device for calculating a distance between eyes and the eyes' target in accordance with this disclosure;

FIG. 1C is a schematic view of a third embodiment of the device for calculating a distance between eyes and the eyes' target in accordance with this disclosure;

FIG. 1D is a schematic view of a fourth embodiment of the device for calculating a distance between eyes and the eyes' target in accordance with this disclosure;

FIG. 2A shows schematically a flowchart of a first embodiment of the method for calculating a distance between eyes and the eyes' target in accordance with this disclosure;

FIG. 2B shows schematically a flowchart of a second embodiment of the method for calculating a distance between eyes and the eyes' target in accordance with this disclosure;

FIG. 3A illustrates schematically the cumulative distribution function (CDF) of the training set of the first embodiment in accordance with this disclosure; and

FIG. 3B illustrates schematically the cumulative distribution function (CDF) of the verifying set of the first embodiment in accordance with this disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

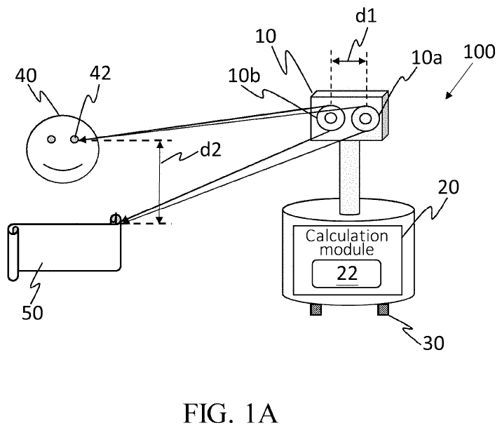

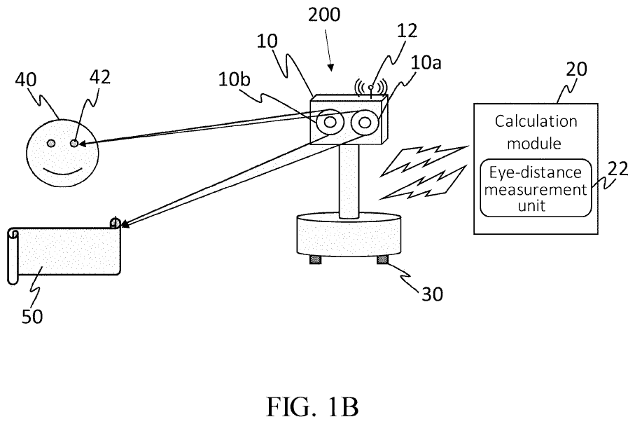

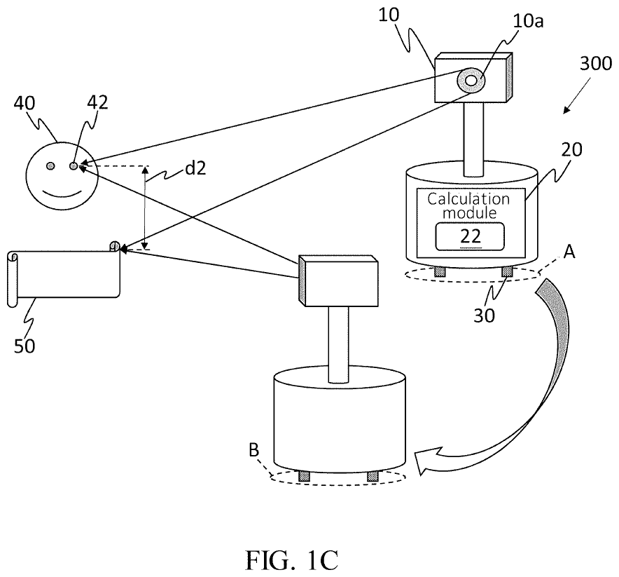

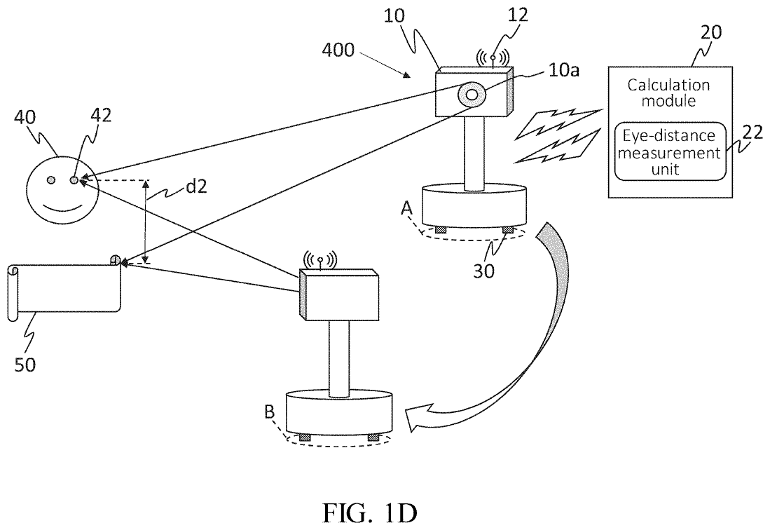

Firstly, referring to FIG. 1A, a schematic view of a first embodiment of the device for calculating a distance between eyes and the eyes' target in accordance with this disclosure is shown. In this embodiment, the device for calculating a distance between eyes and the eyes' target 100 includes a movable image capture module 10 and a calculation module 20.

The movable image capture module 10 is configured to image a test subject 40 and the eyes' target 50 so as to obtain a set of image-capturing data. This set of image-capturing data includes a first-capturing image data and a second-capturing image data. As shown, the movable image capture module 10 has a first camera 10a and a second camera 10b, in which the first camera 10a is spaced from the second camera 10b by a distance d1 (preferably, from a center to another center as shown). The first camera 10a and the second camera 10b are applied simultaneously to image the test subject 40 and the eyes' target 50. The first camera 10a would generate a first-capturing image data while the second camera 10b generates a second-capturing image data. Further, due to the distance-d1 spacing, the resulted first-capturing image data and second-capturing image data would form an angular or linear difference. In other words, the movable image capture module 10 in the first embodiment utilizes double cameras to image simultaneously both the test subject 40 and the eyes' target 50.

The calculation module 20 has an eye-distance measurement unit 22. The calculation module 20 and the movable image capture module 10 are electrically connected with each other. The calculation module 20 is configured to receive the first-capturing image data and the second-capturing image data generated by the movable image capture module 10. Practically, the eye-distance measurement unit 22 can include memories, hard discs, and any data-storing medium the like. In some other embodiments, the eye-distance measurement unit 22 can be isolated from the calculation module 20, and can be constructed in a data-storing module.

The calculation module 20 is configured to utilize the eye-distance measurement unit 22 to analyze the first-capturing image data and the second-capturing image data, and so a set of three-dimensional coordinate values corresponding to the eyes 42 and the eyes' target 50 of the test subject 40 can be determined. Based on this set of three-dimensional coordinate values, the calculation module 20 can derive a distance d2 between the eyes 42 and the eyes' target 50. In this disclosure, the eyes' target 50 can be a notebook computer, a tablet computer, a smart phone, a book, or a TV set.

For example, the eye-distance measurement unit 22 can utilize a convolutional neural network algorithm to analyze the first-capturing image data and the second-capturing image data. Then, as the eye-distance measurement unit 22 obtains image characteristics from the first-capturing image data and the second-capturing image data, so the image characteristics corresponding to the eyes 42 and the eyes' target 50 can be obtained as well. Based on the image characteristics of the eyes 42 and the eyes' target 50, the calculation module 20 can utilize the eye-distance measurement unit 22 to perform relative calculation and estimation so as to derive the distancing among the test subject 40, the eyes 42, the eyes' target 50 and the movable image capture module 10.

Then, the calculation module 20 further utilizes the eye-distance measurement unit 22 to perform estimation through the image characteristics of the test subject 40, the eyes 42, the eyes' target 50, so as to obtain three-dimensional coordinate values of the test subject 40, the eyes 42 and the eyes' target 50 with respect to the movable image capture module 10. Finally, based on the obtained three-dimensional coordinate values, the calculation module 20 can derive the distance d2 between the eyes 42 and the eyes' target 50. In addition, the aforesaid convolutional neural network algorithm can be the fully convolutional neural network (FCN), the region-based convolutional neural network (R-CNN), the fast region-based convolutional neural network (Fast R-CNN), or the VGG16.

Namely, the first embodiment of this disclosure utilizes the calculation module 20 and the eye-distance measurement unit 22 to process at least two photo shots (i.e., the images) so as to determine three-dimensional positions of the test subject 40, the eyes 42 and the eyes' target 50, and further to estimate the distance d2. Such a distance d2 can be a reference for alerting the eyes' distancing, for correcting an ill eyes' hobbit, and also for being stored as a record for proper domestic eyes' behavior.

That is, in the first embodiment of this disclosure, at least one image data of the eyes 42 and the eyes' target 50 corresponding to at least one train object is inputted into the calculation module 20 as training data for the eye-distance measurement unit 22. For example, the calculation module 20 would have 80% of the image data in the eye-distance measurement unit 22 to form a training set, and 20% thereof as a verifying set. When model training of the eye-distance measurement unit 22 reaches a predetermined accuracy bound, then the three-dimensional positions of the eyes 42 of the test subject 40 and the eyes' target 50 can be derived. Further, by applying the equations of Euclidean distance, the distance d2 between the eyes 42 of the test subject 40 and the eyes' target 50 can be determined.

More practically, after the image data is inputted into the eye-distance measurement unit 22, the convolutional neural network algorithm such as the VGG16 can be applied to abstract the corresponding image characteristics as well as the respective object feature map. Then, another convolutional neural network algorithm such as the fast R-CNN can be applied to obtain the position and dimension information of the eyes 42 and the eyes' target 50, and to locate characteristics of the eyes 42 and the eyes' target 50 from the object feature map. Each of the characteristics of the eyes' target 50 is inputted into the depth regressor so as to estimate relative depths among the eye 42, the eyes' target 50 and the movable image capture module 10. Then, the estimated depths would be transmitted to a 3D keypoint regressor, and each of the characteristics of the eyes' target 50 is inputted into the 3D keypoint regressor so as to estimate the three-dimensional position of the eyes' target 50 with respect to the movable image capture module 10. Further, by applying the equations of Euclidean distance, the distance d2 between the eyes 42 of the test subject 40 and the eyes' target 50 can be determined.

In addition, the movable image capture module 10 has a bottom furnished with universal movable wheels 30 for enabling the movable image capture module 10 to move from point A to point B, such that the image-capturing angle or distance can be adjusted to prevent any obstacle from blocking the first camera 10a and the second camera 10b to image the test subject 40 and the eyes' target 50.

Referring to FIG. 1B, a schematic view of a second embodiment of the device for calculating a distance between eyes and the eyes' target in accordance with this disclosure is shown. A difference between the device for calculating a distance between eyes and the eyes' target 200 of this second embodiment and that 100 of the first embodiment is that, in this embodiment, the device 200 further includes a wireless communication module 12 electrically connected with the first camera 10a and the second camera 10b. The wireless communication module 12 is configured to transmit and input, in a wireless manner, the first-capturing image data and the second-capturing image data to a far-end or cloud calculation module 20 and eye-distance measurement unit 22. Except that, all the other components and structures of the second embodiment are resembled to those of the first embodiment, and thus detail thereabout would be omitted herein.

In this disclosure, the wireless communication module 12 can be structured according to the modern mobile communication technology such as the fourth generation of mobile phone mobile communication technology standards (4G), the 5th generation of mobile phone mobile communication technology standards (5G), or the WiFi data transmission technology.

With the calculation module 20 to be built separately, manufacturing cost of the device for calculating a distance between eyes and the eyes' target 200 of the second embodiment can be significantly reduced. In some embodiments of his disclosure, a plurality of the devices 200 can be commonly supported by a unique calculation module 20. Namely, image data of these devices 200 would be provided to the same calculation module 20 for calculating and analyzing the eyes' distancing. In such an application, each of the devices 200 would be assigned an individual identification label to be marked into the image data, such that the device 200 for transmitting the image data can be identified.

Referring to FIG. 1C, a schematic view of a third embodiment of the device for calculating a distance between eyes and the eyes' target in accordance with this disclosure is shown. A difference between the device for calculating a distance between eyes and the eyes' target 300 of this third embodiment and that 100 of the first embodiment is that, in this embodiment, the movable image capture module 10 is equipped with a single camera, i.e., the first camera 10a. Thus, prior to capturing the second-capturing image data, the device 300 shall be moved from the first position A to the second position B, such that the capturing of the second-capturing image data can be performed. Except that, all the other components and structures of the third embodiment are resembled to those of the first embodiment, and thus detail thereabout would be omitted herein.

In the third embodiment, since the movable image capture module 10 has only the first camera 10a for imaging the test subject 40 and the eyes' target 50 to obtain the first-capturing image data, thus, to image the test subject 40 and the eyes' target 50 again for capturing the second-capturing image data, the imaging angle and/or the imaging distance shall be adjusted so as to obtain a different image data of the test subject 40 and the eyes' target 50. In the operation, the movable image capture module 10 at the first position A firstly utilizes the first camera 10a to image the test subject 40 and the eyes' target 50 for obtaining the first-capturing image data, then the device 300 is moved, via the wheels 30, to the second position B for providing different imaging angle and distance, and thus the first camera 10a can image the test subject 40 and the eyes' target 50 there for obtaining the second-capturing image data. Namely, the device 300 of the third embodiment can still perform the desired capturing upon the test subject 40 and the eyes' target 50 by the movable image capture module 10 with one single camera 10a.

Similarly, the calculation module 20 can apply the eye-distance measurement unit 22 to analyze the first-capturing image data and the second-capturing image data so as to realize the three-dimensional coordinate values corresponding to the eyes 42 of the test subject 40 and the eyes' target 50. Then, based on the three-dimensional coordinate values, the calculation module 20 can calculate the distance d2 between the eyes 42 and the eyes' target 50. In some embodiments of this disclosure, the calculation module 20 can further analyze displacements and moving directions of the movable image capture module 10 to derive the distance d2 between the eyes 42 of the test subject 40 and the eyes' target 50.

Referring to FIG. 1D, a schematic view of a fourth embodiment of the device for calculating a distance between eyes and the eyes' target in accordance with this disclosure is shown. A difference between the device for calculating a distance between eyes and the eyes' target 400 of this fourth embodiment and that 200 of the second embodiment is that, in this embodiment, the movable image capture module 10 is equipped only with a single camera; the first camera 10a for example. Thus, prior to capturing the second-capturing image data, the device 300 shall be displaced from the first position A to the second position B, such that the capturing of the second-capturing image data can be performed. Except that, all the other components and structures of the fourth embodiment are resembled to those of the second embodiment, and thus detail thereabout would be omitted herein.

In this embodiment, the movable image capture module 10 at the first position A firstly utilizes the first camera 10a to image the test subject 40 and the eyes' target 50 for obtaining the first-capturing image data, then the device 400 is displaced, via the wheels 30, to the second position B from the position A, and thus the first camera 10a can image the test subject 40 and the eyes' target 50 again for obtaining the second-capturing image data. It shall be noted that, in order to emphasize the importance of the displacement of the movable image capture module 10 in the third and fourth embodiments, FIG. 1C and FIG. 1D are prepared in a more exaggerated manner. In fact, with difference in angles or distances at the two imaging positions of the first camera 10a for imaging the test subject 40 and the eyes' target 50, then the first-capturing image data and the second-capturing image data would be both valid data.

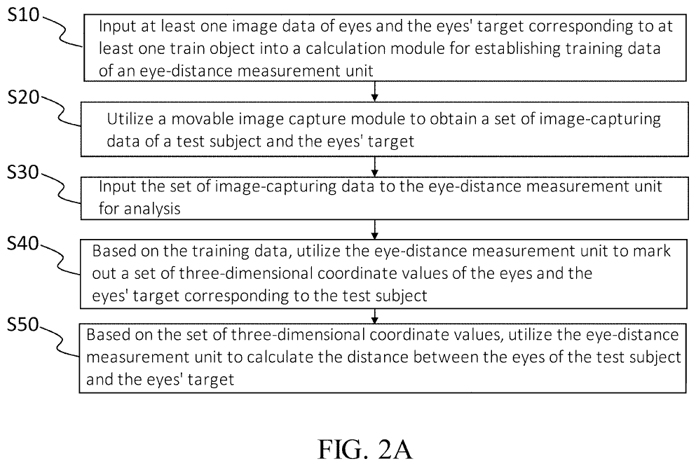

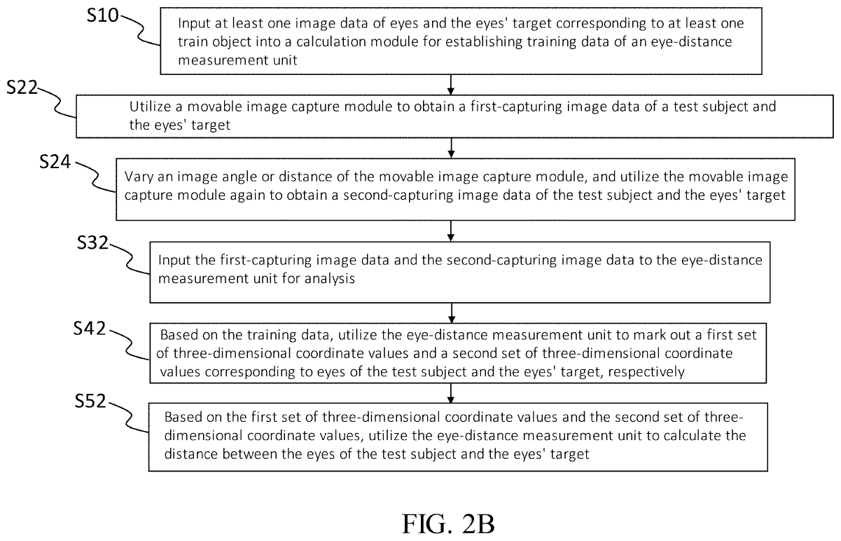

Referring to FIG. 2A, a flowchart of a first embodiment of the method for calculating a distance between eyes and the eyes' target in accordance with this disclosure are listed schematically. As shown in FIG. 1A and FIG. 2A, in step S10, input at least one image data of eyes 42 and the eyes' target 50 corresponding to at least one train object into the calculation module 20 for establishing training data of the eye-distance measurement unit 22.

In step S20, obtain a set of image-capturing data of the test subject 40 and the eyes' target 50 captured by the movable image capture module 10. This set of image-capturing data includes a first-capturing image data and a second-capturing image data. The first-capturing image data and the second-capturing image data are obtained through different angling and distancing. In detail, the first-capturing image data and the second-capturing image data are corresponding to the first imaging angle and the second imaging angle, or the first imaging distance and the second imaging distance, respectively.

In step S30, input the set of image-capturing data into the eye-distance measurement unit 22 for further analysis. The eye-distance measurement unit 22 can adopt a convolutional neural network algorithm to analyze the set of image-capturing data. In some embodiments, the set of image-capturing data can be inputted into the eye-distance measurement unit 22 in a wireless transmission manner.

In step S40, according to the training data, the eye-distance measurement unit 22 marks out a set of three-dimensional coordinate values corresponding to the eyes 42 of the test subject 40 and the eyes' target 50.

In step S50, based on the set of three-dimensional coordinate values, the eye-distance measurement unit 22 can calculate the distance d2 between eyes 42 of the test subject 40 and the eyes' target 50.

Referring to FIG. 2B, a flowchart of a second embodiment of the method for calculating a distance between eyes and the eyes' target in accordance with this disclosure are listed schematically. As shown in FIG. 1C and FIG. 2B, in step S10, input at least one image data of eyes 42 and the eyes' target 50 corresponding to at least one train object into the calculation module 20 for establishing training data of the eye-distance measurement unit 22.

In step S22, obtain a first-capturing image data of the test subject 40 and the eyes' target 50 captured by the movable image capture module 10.

In step S24, vary the image angle or distance of the movable image capture module 10, and obtain a second-capturing image data of the test subject 40 and the eyes' target 50 captured by the movable image capture module 10.

In step S32, input the first-capturing image data and the second-capturing image data into the eye-distance measurement unit 22 for further analysis. In some embodiments, the first-capturing image data and the second-capturing image data can be inputted into the eye-distance measurement unit 22 in a wireless transmission manner.

In step S42, according to the training data, the eye-distance measurement unit 22 marks out a first set of three-dimensional coordinate values (X1,Y1,Z1) and a second set of three-dimensional coordinate values (X2,Y2,Z2) corresponding to the eyes 42 of the test subject 40 and the eyes' target 50.

In step S52, based on the first set of three-dimensional coordinate values and the second set of three-dimensional coordinate values, the eye-distance measurement unit 22 calculates the distance d2 between eyes 42 of the test subject 40 and the eyes' target 50.

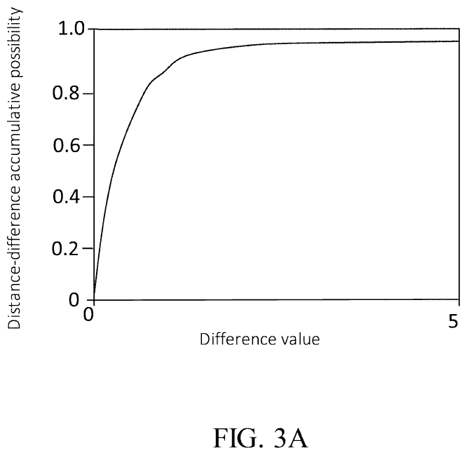

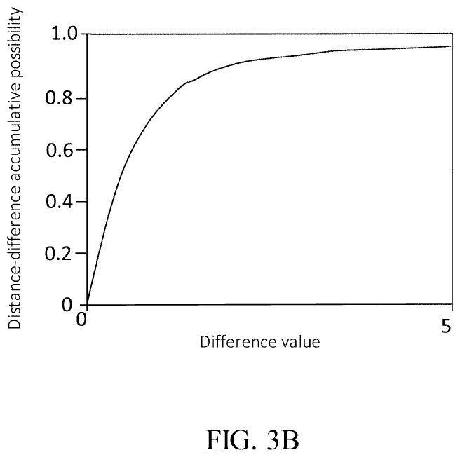

Referring to FIG. 3A, the cumulative distribution function (CDF) of the training set of the first embodiment in accordance with this disclosure is illustrated schematically. In FIG. 3A, the horizontal axis is the difference value (cm), and the vertical axis is the distance-difference accumulative possibility. After being trained by the eye-distance measurement unit 22, the possibility for the difference value less than 5 cm at the distance d2 between the eyes 42 and the eyes' target 50 would reach about 99%.

Referring to FIG. 3B, the cumulative distribution function (CDF) of the verifying set of the first embodiment in accordance with this disclosure is illustrated schematically. In FIG. 3B, the horizontal axis is the difference value (cm), and the vertical axis is the distance-difference accumulative possibility. After being trained by the eye-distance measurement unit 22, the possibility for the difference value less than 5 cm at the distance d2 between the eyes 42 and the eyes' target 50 would reach about 99%.

In summary, the method and device for calculating a distance between eyes and the eyes' target of this disclosure can record the eye-distancing situations of the test object in a more objective manner, so as further for a reference toward modifying the therapy strategy.

According to one embodiment of the method and device for calculating a distance between eyes and the eyes' target, the wireless communication module is introduced to transmit the image data to the far-end or cloud calculation module and the eye-distance measurement unit, such that the cost of the device for calculating a distance between eyes and the eyes' target can be substantially reduced.

According to one embodiment of the method and device for calculating a distance between eyes and the eyes' target, through the training of the eye-distance measurement unit, the possibility for the difference value less than 5 cm would reach 99%, and thus higher measurement accuracy would be obtained.

According to one embodiment of the method and device for calculating a distance between eyes and the eyes' target, no additional wearing mark or sensor is required for the test object, and thus no discomfort or inconvenience would be induced to bother the test object.

With respect to the above description then, it is to be realized that the optimum dimensional relationships for the parts of the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present disclosure.

What is claimed is:

1. A method for calculating a distance between eyes and the eyes' target, comprising the steps of:
inputting at least one image data of eyes and the eyes' target corresponding to at least one train object into a calculation module for establishing training data of an eye-distance measurement unit;
utilizing a movable image capture module to obtain a set of image-capturing data of a test subject and the eyes' target;
inputting the set of image-capturing data to the eye-distance measurement unit for analysis;
based on the training data, utilizing the eye-distance measurement unit to mark out a set of three-dimensional coordinate values of the eyes and the eyes' target corresponding to the test subject; and
based on the set of three-dimensional coordinate values, utilizing the eye-distance measurement unit to calculate the distance between the eyes of the test subject and the eyes' target.

2. The method for calculating a distance between eyes and the eyes' target of claim 1, wherein the set of image-capturing data includes a first-capturing image data and a second-capturing image data, and the first-capturing image data and the second-capturing image data have a first imaging angle and a second imaging angle or a first imaging distance and a second imaging distance, respectively.

3. The method for calculating a distance between eyes and the eyes' target of claim 1, wherein the eye-distance measurement unit adopts a convolutional neural network algorithm to analyze the set of image-capturing data.

4. The method for calculating a distance between eyes and the eyes' target of claim 1, wherein the set of image-capturing data is inputted to the eye-distance measurement unit in a wireless transmission manner.

5. The method for calculating a distance between eyes and the eyes' target of claim 1, wherein the movable image capture module utilizes dual cameras to image the test subject and the eyes' target.

6. A method for calculating a distance between eyes and the eyes' target, comprising the steps of:
inputting at least one image data of eyes and the eyes' target corresponding to at least one train object into a calculation module for establishing training data of an eye-distance measurement unit;
utilizing a movable image capture module to obtain a first-capturing image data of a test subject and the eyes' target;
varying an image angle or distance of the movable image capture module, and utilizing the movable image capture module again to obtain a second-capturing image data of the test subject and the eyes' target;
inputting the first-capturing image data and the second-capturing image data to the eye-distance measurement unit for analysis;
based on the training data, utilizing the eye-distance measurement unit to mark out a first set of three-dimensional coordinate values and a second set of three-dimensional coordinate values corresponding to eyes of the test subject and the eyes' target, respectively; and
based on the first set of three-dimensional coordinate values and the second set of three-dimensional coordinate values, utilizing the eye-distance measurement unit to calculate the distance between the eyes of the test subject and the eyes' target.

7. The method for calculating a distance between eyes and the eyes' target of claim 6, wherein the eye-distance measurement unit adopts a convolutional neural network algorithm to analyze the first-capturing image data and the second-capturing image data.

8. The method for calculating a distance between eyes and the eyes' target of claim 6, wherein the first-capturing image data and the second-capturing image data are inputted to the eye-distance measurement unit in a wireless transmission manner.

9. The method for calculating a distance between eyes and the eyes' target of claim 6, wherein the movable image capture module utilizes a camera to image the test subject and the eyes' target.

10. A device for calculating a distance between eyes and the eyes' target, comprising:
a movable image capture module, configured for imaging a test subject and the eyes' target to obtain a set of image-capturing data; and
a calculation module, having an eye-distance measurement unit, configured for utilizing the eye-distance measurement unit to analyze the set of image-capturing data to further mark out a set of three-dimensional coordinate values corresponding to an eye of the test subject and the eyes' target, the eye-distance measurement unit evaluating the set of three-dimensional coordinate values to calculate the distance between the eye of the test subject and the eyes' target;
wherein at least one image data of the eyes and the eyes' target corresponding to at least one train object is inputted into the calculation module for establishing training data of the eye-distance measurement unit.

11. The device for calculating a distance between eyes and the eyes' target of claim 10, wherein the set of image-capturing data includes a first-capturing image data and a second-capturing image data, and the first-capturing image data and the second-capturing image data have a first imaging angle and a second imaging angle or a first imaging distance and a second imaging distance, respectively.

12. The device for calculating a distance between eyes and the eyes' target of claim 10, wherein the eye-distance measurement unit adopts a convolutional neural network algorithm to analyze the set of image-capturing data.

13. The device for calculating a distance between eyes and the eyes' target of claim 10, wherein the movable image capture module further includes a wireless communication module configured for inputting the set of image-capturing data to the eye-distance measurement unit in a wireless transmission manner.

14. The device for calculating a distance between eyes and the eyes' target of claim 10, wherein the movable image capture module utilizes dual cameras to image the test subject and the eyes' target.

15. A device for calculating a distance between eyes and the eyes' target, comprising:
a movable image capture module, configured for imaging a test subject and the eyes' target to obtain a first-capturing image data, and further to obtain a second-capturing image data of the test subject and the eyes' target after varying an image angle or distance of the movable image capture module; and
a calculation module, having a eye-distance measurement unit, configured for utilizing the eye-distance measurement unit to analyze the first-capturing image data and the second-capturing image data to further mark out a first set of three-dimensional coordinate values and a second set of three-dimensional coordinate values corresponding to eyes of the test subject and the eyes' target, the eye-distance measurement unit evaluating the first set of three-dimensional coordinate values and the second set of three-dimensional coordinate values to calculate the distance between the eyes of the test subject and the eyes' target;
wherein at least one image data of the eyes and the eyes' target corresponding to at least one train object is inputted into the calculation module for establishing training data of the eye-distance measurement unit.

16. The device for calculating a distance between eyes and the eyes' target of claim 15, wherein the eye-distance measurement unit adopts a convolutional neural network algorithm to analyze the first-capturing image data and the second-capturing image data.

17. The device for calculating a distance between eyes and the eyes' target of claim 15, wherein the movable image capture module further includes a wireless communication module configured for inputting the first-capturing image data and the second-capturing image data to the eye-distance measurement unit in a wireless transmission manner.

18. The device for calculating a distance between eyes and the eyes' target of claim 15, wherein the movable image capture module utilizes a camera to image the test subject and the eyes' target.
